(19) United States
(12) Patent Application Publication
(10) Pub. No.: US 2025/0205192 A1
(43) Pub. Date: Jun. 26, 2025

(54) METHODS AND MATERIALS FOR TREATING PANCREATIC DISEASES AND DISORDERS

(71) Applicant: MAYO FOUNDATION FOR MEDICAL EDUCATION AND RESEARCH, Rochester, MN (US)

(72) Inventor: Vijay P. Singh, Scottsdale, AZ (US)

(21) Appl. No.: 18/848,249

(22) PCT Filed: Mar. 31, 2023
(86) PCT No.: PCT/US2023/065200

§ 371 Date: Sep. 18, 2024

Related U.S. Application Data
(60) Provisional application No. 63/325,680, filed on Mar. 31, 2022.

(51) Int. Cl. A61K 31/365; A61P 1/18

(52) U.S. Cl. CPC A61K 31/365; A61P 1/18

(57) ABSTRACT

This document relates to methods and materials for treating pancreatitis (e.g., chronic pancreatitis) in a mammal (e.g., a human). For example, methods for using one or more lipase inhibitors to treat a mammal having or at risk of developing pancreatitis (e.g., chronic pancreatitis) are provided.

### STATEMENT OF PRIORITY

This application claims the benefit of U.S. Provisional Application Ser. No. 63/325,680, filed Mar. 31, 2022, the entire contents of which are incorporated by reference herein.

### TECHNICAL FIELD

This document relates to methods and materials for treating pancreatic diseases, disorders, and conditions in a mammal (e.g., a human). For example, this document provides methods and materials for using one or more lipase inhibitors to treat a mammal having or at risk of developing one or more pancreatic diseases, disorders, and/or conditions.

### BACKGROUND INFORMATION

New cases of chronic pancreatitis develop in about 8 per 100,000 people a year and currently affect about 50 per 100,000 people in the United States (Muniraj et al., Disease-a-Month: DM (2014) 60:530-50). Globally, in 2013, pancreatitis resulted in 123,000 deaths up from 83,000 deaths in 1990 (GBD 2013 Mortality and Causes of Death, Collaborators Lancet (2014) 385:117-71).

Post-operative pancreatic fistula occurs in 10-20% of patients after pancreatic surgery and is a significant cause of morbidity. Post-operative pancreatic fistula has no treatment.

### SUMMARY

This document provides methods and materials for treating pancreatic diseases, disorders, and conditions in a mammal (e.g., a human). For example, this document provides methods and materials for administering one or more lipase inhibitors to a mammal (e.g., a human) having or at risk of developing pancreatic diseases, disorders, and conditions in order to treat the mammal. As described herein, lipase inhibitors can be effective to treat pancreatic diseases and disorders (e.g., pancreatic diseases and disorders associated with adhesions and/or fibrosis). In some cases, one or more lipase inhibitors can be used to reduce or slow the progression of pancreatitis (e.g., chronic pancreatitis), pancreatic cancer, and/or post-operative pancreatic fistulas within a mammal (e.g., a human). In some cases, one or more lipase inhibitors can be used to reduce the risk of developing pancreatitis (e.g., chronic pancreatitis), pancreatic cancer, and/or post-operative pancreatic fistulas within a mammal (e.g., a human). For example, as demonstrated herein, one or more lipase inhibitors can be used to reduce the adhesions in the pancreas (e.g., adhesions between pancreatic acinar cells and/or endothelial cells) and/or in the peritoneal cavity, and/or to reduce fibrosis in the pancreas and the tissues surrounding the pancreas and/or in the peritoneal cavity.

In general, one aspect of this document features methods for reducing adhesion in a pancreas and/or peritoneal cavity of a mammal. The methods can comprise, consist essentially of, or consist of administering a lipase inhibitor to a mammal in need thereof. The mammal can have chronic pancreatitis, pancreatic cancer, or a post-operative pancreatic fistula. The mammal can be a human. The inhibitor can be compound 767 or a pharmaceutically acceptable salt thereof. The inhibitor can have the structure:

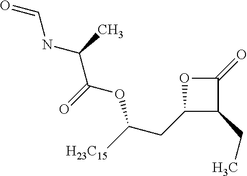

or a pharmaceutically acceptable salt thereof.

In another aspect, this document features methods for reducing fibrosis in a pancreas and/or in the peritoneal cavity of a mammal. The methods can comprise, consist essentially of, or consist of administering a lipase inhibitor to a mammal in need thereof. The mammal can have chronic pancreatitis, pancreatic cancer, or a post-operative pancreatic fistula. The mammal can be a human. The inhibitor can be compound 767 or a pharmaceutically acceptable salt thereof. The inhibitor can have the structure:

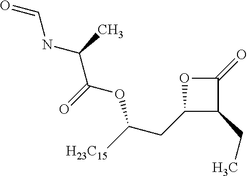

or a pharmaceutically acceptable salt thereof.

In another aspect, this document features methods for reducing inflammation in a pancreas and/or in the peritoneal cavity of a mammal. The methods can comprise, consist essentially of, or consist of administering a lipase inhibitor to a mammal in need thereof. The mammal can have chronic pancreatitis, pancreatic cancer, or a post-operative pancreatic fistula. The mammal can be a human. The inhibitor can be compound 767 or a pharmaceutically acceptable salt thereof. The inhibitor can have the structure:

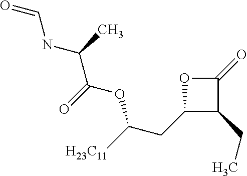

or a pharmaceutically acceptable salt thereof.

In another aspect, this document features uses of a composition comprising a lipase inhibitor to reduce adhesion and/or fibrosis in a pancreas and/or peritoneal cavity of a mammal. The mammal can be a human. The inhibitor can be compound 767 or a pharmaceutically acceptable salt thereof. The inhibitor can have the structure:

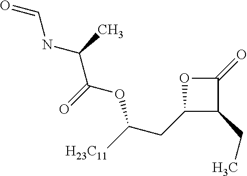

or a pharmaceutically acceptable salt thereof.

In another aspect, this document features lipase inhibitors for use as a medicament to reduce adhesion and/or fibrosis in a pancreas and/or peritoneal cavity of a mammal. The mammal can be a human. The inhibitor can be compound 767 or a pharmaceutically acceptable salt thereof. The inhibitor can have the structure:

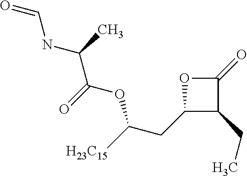

or a pharmaceutically acceptable salt thereof.

In another aspect, this document features lipase inhibitors for use in reducing adhesion and/or fibrosis in a pancreas and/or peritoneal cavity of a mammal. The mammal can be a human. The inhibitor can be compound 767 or a pharmaceutically acceptable salt thereof. The inhibitor can have the structure:

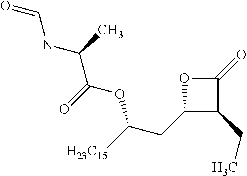

or a pharmaceutically acceptable salt thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1, panels A-C show representative images of peritoneal cavities of control mice (FIG. 1, panel A) and mice treated with glyceryl trilinoleate (GTL; FIG. 1, panels B and C). Mice administered with GTL had nodules and strands (arrows).

FIG. 2, panels A and B show representative images of pancreas duodenum and spleen removed en bloc from control mice (FIG. 2, panel A) and from mice treated with GTL (FIG. 2, panel B). Mice administered with GTL had nodules and strands (arrows) in planes between the organs.

FIG. 3 contains a representative image of a morphologic evaluation of a spleen from a mouse administered with GTL. Staining shows extensive fibrosis like appearance in the area between the pancreas and spleen.

FIG. 4, panels A-C contain representative images of nodules from mice treated with GTL that were stained with H&E (FIG. 4, panel A), von Kossa stain (FIG. 4, panel B), or Sirius red (FIG. 4, panel C). Fibrosis surrounds partially hydrolyzed lipid in mice that were administered GTL.

FIG. 5, panels A-B contain representative images of nodules from mice treated with GTL that were stained with von Kossa stain (FIG. 5, panel A) or Masson's trichrome (FIG. 5, panel B). Fibrosis surrounds partially hydrolyzed lipid in mice that were administered GTL.

FIG. 6, panels A-B contain representative images of organs from the peritoneal cavity (upper panels) and around the pancreas (lower panels) from mice treated with GTL only (FIG. 6, panel A) and from mice treated with both GTL and 767 (FIG. 6, panel B). The GTL group had clearly discernable nodules (FIG. 6, panel A, arrows). The mice treated with both GTL and 767 did not have noticeable nodules in the peritoneal cavity or in the vicinity of the pancreas.

FIG. 7, panels A-B. FIG. 7, panel A is a graph showing the unsaturated fatty acid (UFA) composition of postoperative pancreatic collections. The black dots denote early collections. FIG. 7, panel B shows a time course of change in non-esterified fatty acid (NEFA) composition and concentrations in micromolar (μM) of post pancreatic surgery collections. The black dots have the highest NEFA.

### DETAILED DESCRIPTION

This document provides methods and materials for treating pancreatic diseases, disorders, and conditions. For example, this document provides methods and materials for administering one or more lipase inhibitors to a mammal (e.g., a human) having or at risk of developing one or more pancreatic diseases, disorders, and/or conditions to treat the mammal. In some cases, one or more lipase inhibitors can be used to reduce or slow the progression of pancreatitis (e.g., chronic pancreatitis), pancreatic cancer, and/or post-operative pancreatic fistulas. For example, one or more lipase inhibitors can be administered to a mammal having pancreatitis (e.g., chronic pancreatitis), pancreatic cancer, and/or post-operative pancreatic fistulas to reduce or slow the progression of the pancreatitis (e.g., chronic pancreatitis), pancreatic cancer, and/or post-operative pancreatic fistulas. In some cases, one or more lipase inhibitors can be used to reduce the risk of developing pancreatitis (e.g., chronic pancreatitis), pancreatic cancer, and/or post-operative pancreatic fistulas. For example, one or more lipase inhibitors can be administered to a mammal at risk of developing pancreatitis to reduce the risk of developing pancreatitis (e.g., chronic pancreatitis), pancreatic cancer, and/or post-operative pancreatic fistulas.

The methods provided herein can be used to treat any appropriate pancreatic disease, disorder, or condition. Examples of pancreatic diseases, disorders, and conditions that can be treated as described herein include, without limitation, pancreatitis, pancreatic cancer, post-operative pancreatic fistulas, and cystic lesions of the pancreas. In some embodiments, the pancreatic disease, disorder, or condition is not acute pancreatitis. When treating pancreatitis as described herein, the pancreatitis can be chronic pancreatitis. The chronic pancreatitis can be due to heavy alcohol use and/or can develop from acute pancreatitis. When treating pancreatic cancer as described herein, the pancreatic cancer can be any stage of pancreatic cancer. In some cases when treating a pancreatic cancer as described herein, the pancreatic cancer can include fibrotic stroma.

In some cases, one or more (e.g., one, two, three, four, or more) lipase inhibitors can be used to reduce the severity of one or more symptoms of pancreatitis (e.g., chronic pancreatitis). For example, one or more lipase inhibitors can be administered to a mammal (e.g., a human) in need thereof (e.g., a human having or at risk of developing pancreatitis such as chronic pancreatitis) to reduce the severity of one or more symptoms of the pancreatitis. Examples of symptoms of pancreatitis (e.g., chronic pancreatitis) include, without limitation, upper abdominal pain, abdominal pain that feels worse after eating, losing weight without trying, and steatorrhea. In some cases, the methods and materials described herein can be effective to reduce the severity of one or more symptoms of pancreatitis (e.g., chronic pancreatitis) in a mammal having pancreatitis (e.g., chronic pancreatitis) by, for example, 10, 20, 30, 40, 50, 60, 70, 80, 90, 95, or more percent.

In some cases, one or more (e.g., one, two, three, four, or more) lipase inhibitors can be used to reduce or eliminate inflammation in the pancreas and/or peritoneal cavity of a mammal (e.g., a human). For example, one or more lipase inhibitors can be administered to a mammal (e.g., a human) in need thereof (e.g., a human having or at risk of developing one or more pancreatic diseases, disorders, and/or conditions) to reduce or eliminate inflammation in the pancreas and/or peritoneal cavity of the mammal. In some cases, the methods and materials described herein can be effective to reduce inflammation in the pancreas and/or peritoneal cavity of a mammal having or at risk of developing one or more pancreatic diseases, disorders, and/or conditions by, for example, 10, 20, 30, 40, 50, 60, 70, 80, 90, 95, or more percent.

In some cases, one or more (e.g., one, two, three, four, or more) lipase inhibitors can be used to reduce or slow the progression of pancreatitis. For example, one or more lipase inhibitors can be administered to a mammal (e.g., a human) in need thereof (e.g., a human having pancreatitis such as chronic pancreatitis) to reduce or slow the progression of pancreatitis in the mammal. In some cases, the methods and materials described herein can be effective to reduce or slow the progression of pancreatitis in a mammal having pancreatitis (e.g., chronic pancreatitis) by, for example, 10, 20, 30, 40, 50, 60, 70, 80, 90, 95, or more percent. In some cases, the methods and materials described herein can be effective to reduce or slow the progression of pancreatitis in a mammal having pancreatitis (e.g., chronic pancreatitis) by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) lipase inhibitors can be used to delay or prevent the development of pancreatitis. For example, one or more lipase inhibitors can be administered to a mammal (e.g., a human) in need thereof (e.g., a human at risk of developing pancreatitis such as chronic pancreatitis) to delay or prevent the development of pancreatitis in the mammal. In some cases, the methods and materials described herein can be effective to delay the development of pancreatitis in a mammal at risk of developing pancreatitis (e.g., chronic pancreatitis) by, for example, 10, 20, 30, 40, 50, 60, 70, 80, 90, 95, or more percent. In some cases, the methods and materials described herein can be effective to delay the development of pancreatitis in a mammal at risk of developing pancreatitis (e.g., chronic pancreatitis) by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) lipase inhibitors can be used to reduce or eliminate adhesion in the pancreas (e.g., adhesion between pancreatic acinar cells and/or endothelial cells) and/or peritoneal cavity. For example, one or more lipase inhibitors can be administered to a mammal (e.g., a human) in need thereof (e.g., a human having one or more pancreatic diseases, disorders, and/or conditions) to reduce or eliminate adhesion in the pancreas (e.g., adhesion between pancreatic acinar cells and/or endothelial cells) and/or peritoneal cavity of the mammal. In some cases, the methods and materials described herein can be effective to reduce adhesion in the pancreas (e.g., adhesion between pancreatic acinar cells and/or endothelial cells) and/or peritoneal cavity of a mammal having one or more pancreatic diseases, disorders, and/or conditions by, for example, 10, 20, 30, 40, 50, 60, 70, 80, 90, 95, or more percent.

In some cases, one or more (e.g., one, two, three, four, or more) lipase inhibitors can be used to delay or prevent development of adhesion in the pancreas (e.g., adhesion between pancreatic acinar cells and/or endothelial cells) and/or peritoneal cavity. For example, one or more lipase inhibitors can be administered to a mammal (e.g., a human) in need thereof (e.g., a human at risk of developing one or more pancreatic diseases, disorders, and/or conditions) to delay or prevent the development of adhesion in the pancreas (e.g., adhesion between pancreatic acinar cells and/or endothelial cells) and/or peritoneal cavity of the mammal. In some cases, the methods and materials described herein can be effective to delay the development of adhesion in the pancreas (e.g., adhesion between pancreatic acinar cells and/or endothelial cells) and/or peritoneal cavity of a mammal at risk of developing one or more pancreatic diseases, disorders, and/or conditions by, for example, 10, 20, 30, 40, 50, 60, 70, 80, 90, 95, or more percent. In some cases, the methods and materials described herein can be effective to delay the development of adhesion in the pancreas (e.g., adhesion between pancreatic acinar cells and/or endothelial cells) and/or peritoneal cavity of a mammal at risk of developing one or more pancreatic diseases, disorders, and/or conditions by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) lipase inhibitors can be used to reduce or eliminate fibrosis in the pancreas and/or peritoneal cavity. For example, one or more lipase inhibitors can be administered to a mammal (e.g., a human) in need thereof (e.g., a human having one or more pancreatic diseases, disorders, and/or conditions) to reduce or eliminate fibrosis in the pancreas and/or peritoneal cavity of the mammal. In some cases, the methods and materials described herein can be effective to reduce fibrosis in the pancreas and/or peritoneal cavity of a mammal having one or more pancreatic diseases, disorders, and/or conditions by, for example, 10, 20, 30, 40, 50, 60, 70, 80, 90, 95, or more percent.

In some cases, one or more (e.g., one, two, three, four, or more) lipase inhibitors can be used to delay or prevent development of fibrosis in the pancreas and/or in the peritoneal cavity. For example, one or more lipase inhibitors can be administered to a mammal (e.g., a human) in need thereof (e.g., a human at risk of developing one or more pancreatic diseases, disorders, and/or conditions) to delay or prevent the development of fibrosis in the pancreas and/or in the peritoneal cavity of the mammal. In some cases, the methods and materials described herein can be effective to delay the development of fibrosis in the pancreas and/or in the peritoneal cavity of a mammal at risk of developing one or more pancreatic diseases, disorders, and/or conditions by, for example, 10, 20, 30, 40, 50, 60, 70, 80, 90, 95, or more percent. In some cases, the methods and materials described herein can be effective to delay the development of fibrosis in the pancreas and/or in the peritoneal cavity of a mammal at risk of developing one or more pancreatic diseases, disorders, and/or conditions by, for example, at least 6 months (e.g., about 6 months, about 8 months, about 10 months, about 1 year, about 1.5 years, about 2 years, about 2.5 years, about 3 years, about 4 years, about 5 years, or more).

In some cases, one or more (e.g., one, two, three, four, or more) lipase inhibitors can be used to improve survival of a mammal. For example, one or more lipase inhibitors can be administered to a mammal (e.g., a human) in need thereof (e.g., a human having one or more pancreatic diseases, disorders, and/or conditions) to improve survival of the mammal. In some cases, the methods and materials described herein can be effective to improve survival of a mammal having one or more pancreatic diseases, disorders, and/or conditions by, for example, 10, 20, 30, 40, 50, 60, 70, 80, 90, 95, or more percent. In some cases, the methods and materials described herein can be effective to improve survival of a mammal having one or more pancreatic diseases, disorders, and/or conditions by, for example, at least 6 months (e.g., about 6 months, about 8 months, about 10 months, about 1 year, about 1.5 years, about 2 years, about 2.5 years, about 3 years, about 4 years, about 5 years, or more).

Any type of mammal having or at risk for developing one or more pancreatic diseases, disorders, and/or conditions can be treated as described herein. Examples of mammals that can have or be at risk for developing one or more pancreatic diseases, disorders, and/or conditions and can be treated as described herein include, without limitation, humans, non-human primates such as monkeys, dogs, cats, horses, cows, pigs, sheep, rabbits, mice, and rats.

In some cases, methods described herein can include identifying a mammal (e.g., a human) as having or being at risk of developing one or more pancreatic diseases, disorders, and/or conditions. Any appropriate method can be used to identify a mammal as having or as being at risk for developing one or more pancreatic diseases, disorders, and/or conditions. For example, abdominal ultrasound, computerized tomography (CT) scan, and/or blood tests (e.g., for an increase in amylase and/or lipase such as a level greater than about threefold the upper limit of normal) can be used to identify a human or other mammal as having or being at risk of developing pancreatitis (e.g., chronic pancreatitis). For example, imaging tests (e.g., CT) scans, magnetic resonance imaging (MRI), positron emission tomography (PET) scans, endoscopic ultrasound (EUS)), pancreatic tissue biopsy, and/or blood tests (e.g., for pancreatic tumor markers such as CA19-9) can be used to identify a human or other mammal as having or being at risk of developing pancreatic cancer. For example, amylase levels (e.g., serum amylase levels) and/or imaging tests (e.g., contrast-enhanced CT and endoscopic retrograde cholangiopancreatography (ERCP)) can be used to identify a human or other mammal as having or being at risk of developing one or more pancreatic fistulas.

A mammal (e.g., a human) having or at risk of developing one or more pancreatic diseases, disorders, and/or conditions can be administered or instructed to self-administer any one or more (e.g., one, two, three, four, or more) lipase inhibitors. A lipase inhibitor can inhibit any appropriate lipase (e.g., a pancreatic lipase). For example, a lipase inhibitor can inhibit pancreatic lipase (PL) polypeptides and/or pancreatic lipase related protein 2 (PLRP2) polypeptides. In some cases, a lipase inhibitor can reduce or eliminate the ability of the lipase to hydrolyze one or more lipids. A lipase inhibitor that can be used as described herein can inhibit a lipase that can hydrolyze any appropriate one or more lipids. In some cases, a lipase inhibitor that can be used to treat a mammal (e.g., a human) having or risk of developing one or more pancreatic diseases, disorders, and/or conditions as described herein can inhibit a lipase that can hydrolyze a triglyceride (e.g., a neutral triglyceride). Examples of lipids that can be hydrolyzed by a lipase that can be inhibited by a lipase inhibitor that can be used to treat a mammal (e.g., a human) having or risk of developing one or more pancreatic diseases, disorders, and/or conditions as described herein include, without limitation, linoleins (e.g., glyceryl trilinoleates). A lipase inhibitor can inhibit lipase polypeptide activity or lipase polypeptide expression. Examples of compounds that can reduce or eliminate polypeptide activity of a lipase include, without limitation, antibodies (e.g., neutralizing antibodies) and small molecules (e.g., 767) that target (e.g., target and bind to) a lipase. When a compound that can reduce or eliminate polypeptide activity of a lipase is a small molecule that targets (e.g., targets and binds) to a lipase, the small molecule can be in the form of a salt (e.g., a pharmaceutically acceptable salt). Examples of compounds that can reduce or eliminate polypeptide expression of a lipase include, without limitation, nucleic acid molecules designed to induce RNA interference of polypeptide expression of a lipase (e.g., a siRNA molecule or a shRNA molecule), antisense molecules, and miRNAs. In some cases, a lipase inhibitor that can be used to treat one or more pancreatic diseases, disorders, and/or conditions as described herein can be 767 or a pharmaceutically acceptable salt thereof and have the following structure:

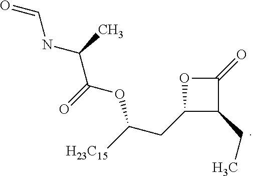

In some cases, a lipase inhibitor that can be used to treat one or more pancreatic diseases, disorders, and/or conditions as described herein can be as described elsewhere (see, e.g., WO 2019/014434).

In some cases, one or more (e.g., one, two, three, four, or more) lipase inhibitors can be formulated into a composition (e.g., a pharmaceutically acceptable composition) for administration to a mammal (e.g., a human) having or at risk of developing one or more pancreatic diseases, disorders, and/or conditions. For example, one or more lipase inhibitors can be formulated together with one or more pharmaceutically acceptable carriers (additives), excipients, and/or diluents. Examples of pharmaceutically acceptable carriers, excipients, and diluents that can be used in a composition described herein include, without limitation, cyclodextrins (e.g., beta-cyclodextrins such as KLEPTOSE®), dimethyl sulfoxide (DMSO), sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline such as phosphate buffered saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil.

In some cases, when a composition containing one or more (e.g., one, two, three, four, or more) lipase inhibitors is administered to a mammal (e.g., a human) having or at risk of developing one or more pancreatic diseases, disorders, and/or conditions, the composition can be designed for oral or parenteral (including, without limitation, subcutaneous, intramuscular, intravenous, intradermal, intra-cerebral, intrathecal, intraabdominal, and intraperitoneal injections (e.g., peripancreatic)) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain antioxidants, buffers, bacteriostats, and solutes that render the formulation isotonic with the blood of the intended recipient.

In some cases, a composition containing one or more (e.g., one, two, three, four, or more) lipase inhibitors can be in the form of a sterile injectable suspension (e.g., a sterile injectable aqueous or oleaginous suspension). This suspension may be formulated using, for example, suitable dispersing or wetting agents (such as, for example, Tween 80) and suspending agents. The sterile injectable preparation can be a sterile injectable solution or suspension in a non-toxic parenterally-acceptable diluent or solvent, for example, as a solution in 1, 3-butanediol. Examples of acceptable vehicles and solvents that can be used include, without limitation, saline, mannitol, water, Ringer's solution, and isotonic sodium chloride solution. In addition, sterile, fixed oils can be used as a solvent or suspending medium. In some cases, a bland fixed oil can be used such as synthetic mono- or di-glycerides.

In some cases, a composition containing one or more (e.g., one, two, three, four, or more) lipase inhibitors can be presented in unit-dose or multi-dose containers, for example, sealed ampules and vials, and may be stored in a freeze dried (lyophilized) condition requiring only the addition of the sterile liquid carrier, for example water for injections, immediately prior to use.

A composition containing one or more (e.g., one, two, three, four, or more) lipase inhibitors can be administered to a mammal (e.g., a human) having or at risk of developing one or more pancreatic diseases, disorders, and/or conditions in any therapeutically effective amount (e.g., any appropriate dose). A therapeutically effective amount of a composition containing one or more lipase inhibitors can be any amount that can treat a mammal having or at risk of developing one or more pancreatic diseases, disorders, and/or conditions as described herein without producing significant toxicity to the mammal. The therapeutically effective amount can remain constant or can be adjusted as a sliding scale or variable dose depending on the mammal's response to treatment. Various factors can influence the actual therapeutically effective amount used for a particular application. For example, the frequency of administration, duration of treatment, use of multiple treatment agents, route of administration, and/or severity of the one or more pancreatic diseases, disorders, and/or conditions in the mammal being treated may require an increase or decrease in the actual therapeutically effective amount administered.

A composition containing one or more (e.g., one, two, three, four, or more) lipase inhibitors can be administered to a mammal (e.g., a human) having or at risk of developing one or more pancreatic diseases, disorders, and/or conditions in any appropriate frequency. The frequency of administration can be any frequency that can treat a mammal having or at risk of developing one or more pancreatic diseases, disorders, and/or conditions without producing significant toxicity to the mammal. For example, the frequency of administration can be from about twice a day to about one every other day, once a day to about once a week, from about once a week to about once a month, or from about twice a month to about once a month. The frequency of administration can remain constant or can be variable during the duration of treatment. As with the therapeutically effective amount, various factors can influence the actual frequency of administration used for a particular application. For example, the therapeutically effective amount, duration of treatment, use of multiple treatment agents, and/or route of administration may require an increase or decrease in administration frequency.

A composition containing one or more (e.g., one, two, three, four, or more) lipase inhibitors can be administered to a mammal (e.g., a human) having or at risk of developing one or more pancreatic diseases, disorders, and/or conditions for any appropriate duration. An effective duration for administering or using a composition containing one or more lipase inhibitors can be any duration that can treat a mammal having or at risk of developing one or more pancreatic diseases, disorders, and/or conditions without producing significant toxicity to the mammal. For example, the effective duration can vary from several weeks to several months, from several months to several years, or from several years to a lifetime. Multiple factors can influence the actual effective duration used for a particular treatment. For example, an effective duration can vary with the frequency of administration, therapeutically effective amount, use of multiple treatment agents, and/or route of administration.

In some cases, methods for treating a mammal (e.g., a human) having or at risk of developing one or more pancreatic diseases, disorders, and/or conditions as described herein (e.g., by administering one or more lipase inhibitors) can include administering to the mammal one or more (e.g., one, two, three, four, or more) lipase inhibitors as the sole active ingredient to treat the mammal. For example, a composition containing one or more lipase inhibitors can include the one or more lipase inhibitors as the sole active ingredient in the composition that is effective to treat a mammal having or at risk of developing one or more pancreatic diseases, disorders, and/or conditions.

In some cases, methods for treating a mammal (e.g., a human) having or at risk of developing one or more pancreatic diseases, disorders, and/or conditions as described herein (e.g., by administering one or more lipase inhibitors) also can include administering to the mammal one or more (e.g., one, two, three, four, five or more) additional agents used to treat one or more pancreatic diseases, disorders, and/or conditions to the mammal and/or performing therapies used to treat one or more pancreatic diseases, disorders, and/or conditions on the mammal. For example, a combination therapy used to treat one or more pancreatic diseases, disorders, and/or conditions can include administering to the mammal (e.g., a human) one or more lipase inhibitors described herein and one or more (e.g., one, two, three, four, five or more) agents used to treat one or more pancreatic diseases, disorders, and/or conditions. Examples of agents that can be administered to a mammal to treat pancreatitis (e.g., chronic pancreatitis) include, without limitation, acetaminophen, ibuprofen, opioids (e.g., codeine and morphine), pancreatic enzymes, anti-inflammatories, and any combinations thereof. Examples of agents that can be administered to a mammal to treat pancreatic cancer include, without limitation, gemcitabine, 5-fluorouracil (5-FU), oxaliplatin, albumin-bound paclitaxel, capecitabine, cisplatin, irinotecan, and any combinations thereof. Examples of agents that can be administered to a mammal to treat pancreatic fistulas include, without limitation, somatostatins, somatostatin analogues, and any combinations thereof. In cases where one or more lipase inhibitors are used in combination with additional agents used to treat one or more pancreatic diseases, disorders, and/or conditions, the one or more additional agents can be administered at the same time (e.g., in a single composition containing both one or more lipase inhibitors and the one or more additional agents) or independently. For example, one or more lipase inhibitors described herein can be administered first, and the one or more additional agents administered second, or vice versa.

In some cases, a combination therapy used to treat one or more pancreatic diseases, disorders, and/or conditions can include administering to the mammal (e.g., a human) one or more (e.g., one, two, three, four, or more) lipase inhibitors described herein and performing one or more (e.g., one, two, three, four, five or more) additional therapies used to treat one or more pancreatic diseases, disorders, and/or conditions on the mammal. Examples of therapies used to treat pancreatitis (e.g., chronic pancreatitis) include, without limitation, endoscopic retrograde cholangiopancreatography, surgical removal of the gallbladder, and/or surgical removal of pancreatic fluid. Examples of therapies used to treat pancreatic cancer include, without limitation, surgery, radiation therapy, surgery to remove one or more tumors within the pancreas, and/or surgical removal of all or part of the pancreas. Examples of therapies used to treat pancreatic fistulas include, without limitation, total parenteral nutrition and/or fistulectomy. In cases where one or more lipase inhibitors described herein are used in combination with one or more additional therapies used to treat one or more pancreatic diseases, disorders, and/or conditions, the one or more additional therapies can be performed at the same time or independently of the administration of one or more lipase inhibitors described herein. For example, one or more lipase inhibitors described herein can be administered before, during, or after the one or more additional therapies are performed.

In certain instances, a course of treatment and the severity of one or more symptoms related to the condition being treated (e.g., one or more pancreatic diseases, disorders, and/or conditions) can be monitored. Any appropriate method can be used to determine whether or not the severity of a symptom is reduced. For example, the severity of a symptom of one or more pancreatic diseases, disorders, and/or conditions can be assessed using abdominal ultrasound, CT scan, and/or blood tests (e.g., for an increase in amylase and/or lipase such as a level greater than about threefold the upper limit of normal) at different time points.

The invention will be further described in the following examples, which do not limit the scope of the invention described in the claims.

### EXAMPLES

### Example 1: Lipase Inhibition to Prevent Adhesions and Chronic Pancreatitis

This Example describes the use of lipase inhibitor 767 (also called RABI-767) to prevent adhesions and/or chronic pancreatitis.

### Materials and Methods

### Reagents and Mice:

25% glyceryl trilinoleate (GTL) homogenous suspension in saline was prepared by heating, mixing, and sonication.

Agent 767 was dissolved in a 25% sonicated suspension of GTL in saline. This was prepared by adding 0.5 of the 25% GTL suspension to 10 mg of 767 at room temperature. The solution was then mixed, heated gently to 37-40° C., and sonicated to ensure homogeneous emulsion.

Each experiment used 4 male CD-1 mice (35-39 grams).

### Groups:

Two mice were each given 0.2 mL of 25% GTL by intraperitoneal injection.

Two mice were given 0.2 mL of 767 in 25% GTL by intraperitoneal injection.

### Endpoint:

Mice were sacrificed after 1 week, and the peritoneal cavity was assessed for nodules.

### Results

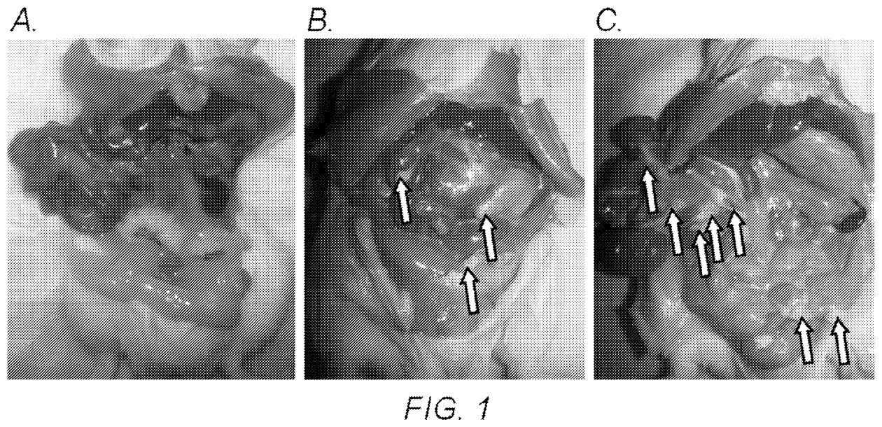

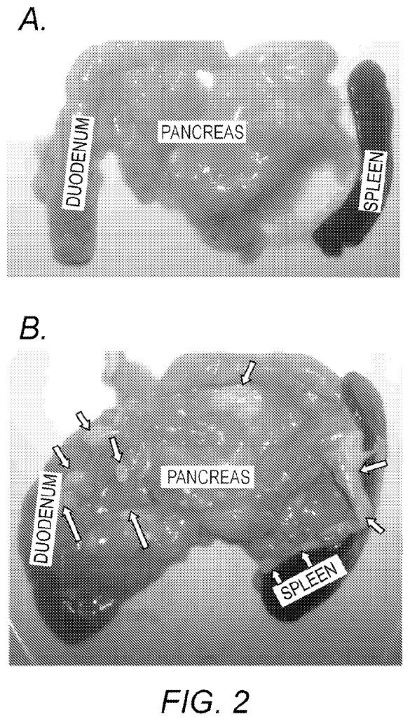

The peritoneal and histologic appearance of mice that received the GTL were examined. The peritoneal cavity of control mice appeared normal (FIG. 1, panel A). The peritoneal cavity of mice given GTL had nodules and strands (FIG. 1, panels B and C). These nodules and strands can also be seen in the en bloc excised pancreas, duodenum, and spleen (FIG. 2). The planes between the duodenum, pancreas, and spleen were well maintained in control mice (FIG. 2, panel A). The organs from mice that received GTL were adherent and difficult to separate. The organs from mice that received GTL contained numerous nodules and strands (FIG. 2, panel B). These appearances are consistent with chronic pancreatitis even without triggering pancreatitis.

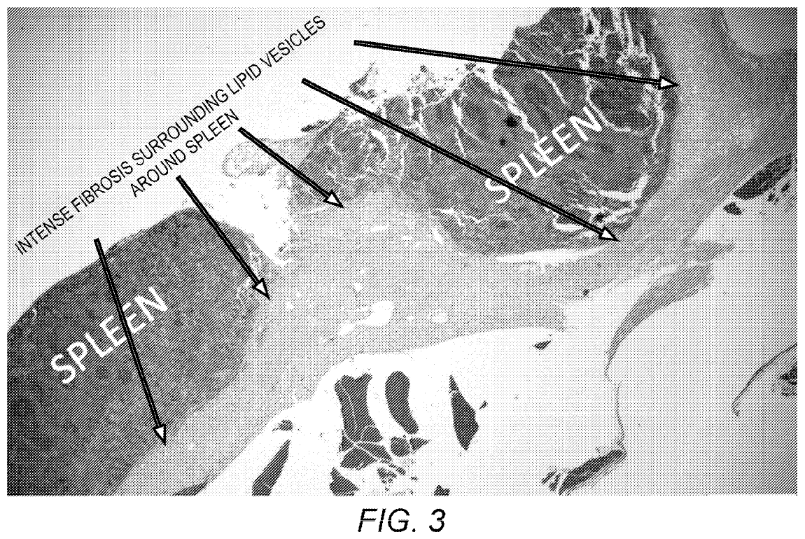

On morphologic evaluation of stained sections from the organs shown in FIG. 2, an extensive fibrosis-like appearance was observed in the area between the pancreas and spleen (FIG. 3). The stained section corresponds to the large strand of fibrosis between the pancreas and spleen in FIG. 2, panel B.

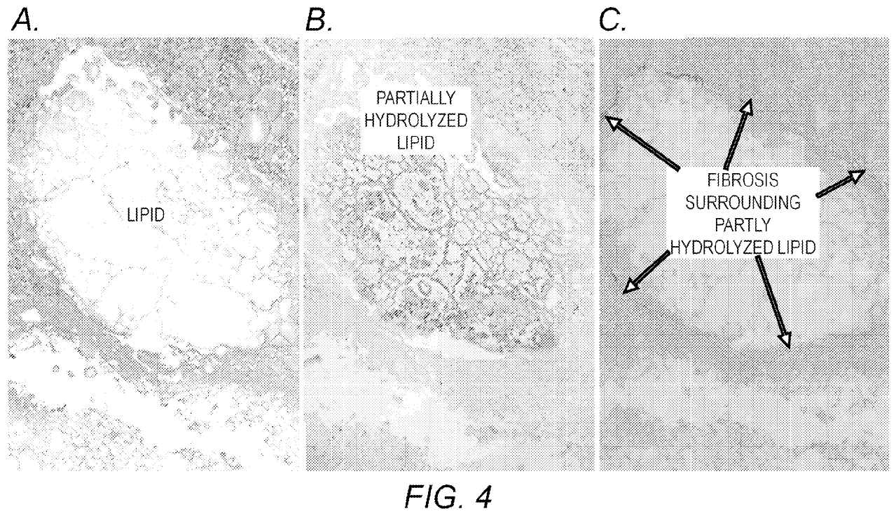

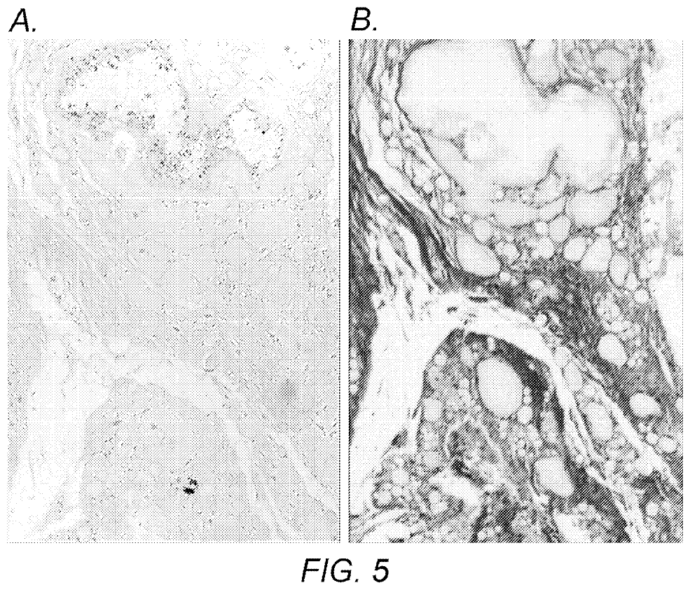

Staining of the nodules with H&E shows partly washed-out material with a lobular appearance (FIG. 4, panel A). The nodules were also stained with von Kossa stain, and the weakly von Kossa-positive globules demonstrate partly partially hydrolyzed GTL (FIG. 4, panel B), even though the mice did not exhibit pancreatitis abnormalities as examined by blood urea nitrogen (BUN), glucose, or albumin levels. The nodules were also stained with Sirius red, and the wall of the nodules were strongly positive for fibrosis (FIG. 4, panel C). Fibrosis was also seen when nodules were stained with Masson's trichrome (FIG. 5, panel B). This fibrosis is adjacent to weakly positive von Kossa areas (asterisks in FIG. 5, panel A).

Gross adhesions and fibrosis surrounding lipid vesicles that were resistant to organic solvents used in histology (e.g., xylene, alcohol) were noted. However, no adhesions surrounding the neutral lipid, that was washed off during tissue processing, were observed. A neutral triglyceride lipid like GTL would normally be washed out of cells such as adipocytes in processing. Therefore, the finding of fibrosis around lipid vesicles, that were not washed off by organic solvents, suggests the low-grade chronic lipolysis of triglyceride results in saponification of the fatty acids so produced and the resulting fibrosis. It was thus examined whether inhibition of intraperitoneal lipolysis could prevent adhesions and chronic pancreatitis.

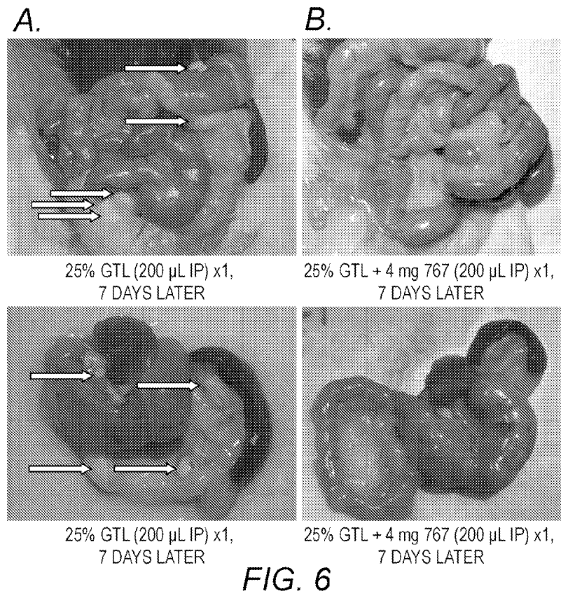

Mice were given GTL with or without 767, and the peritoneal and histologic appearances of the mice were examined. Mice that were given GTL had clearly discernable nodules (FIG. 6, panel A) in the peritoneal cavity (upper panel) and around the pancreas (lower panel). Mice that were given both GTL and 767 did not have noticeable nodules (FIG. 6, panel B) in the peritoneal cavity (upper panel) or in the vicinity of the pancreas (lower panel).

767 also prevented adhesions in the peritoneal cavity in spaces away from the pancreas (FIG. 1, panels B and C; and FIG. 6, panel A).

Together these results demonstrate that one or more lipase inhibitors can be used to treat pancreatitis (e.g., chronic pancreatitis).

### Example 2: Lipase Inhibition and Post-Operative Pancreatic Fistula

This Example describes the use of lipase inhibitor 767 to prevent post-operative pancreatic fistulas.

### Methods

The fatty acid composition and amounts in postoperative pancreatic fluid collections were measured using gas chromatography, mass spectrometry. Fatty acid proportions were characterized as unsaturated (with double bonds) and those that are saturated (without double bonds).

### Results

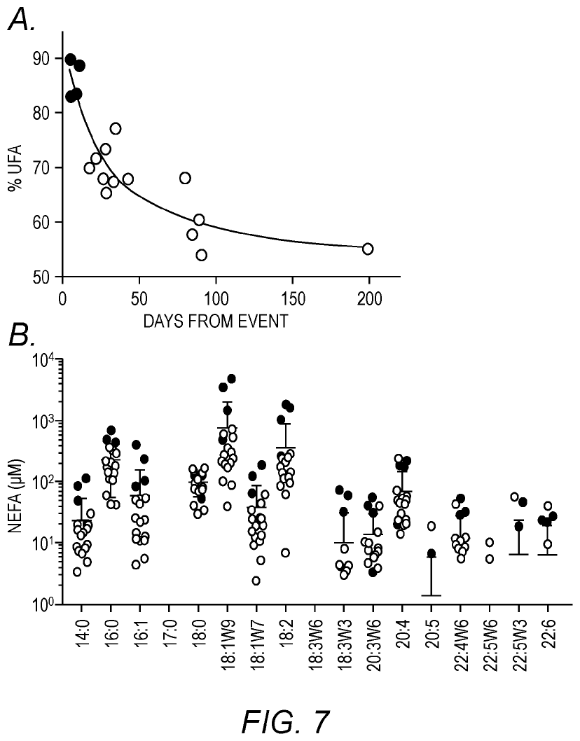

The data are shown in FIG. 7. The 4 black dots in FIG. 7, panel A denote early collections and are the same as the black dots in FIG. 7, panel B. Note that the black dots in FIG. 7, panel B also have the highest NEFA. The amounts of NEFA become less later during the post-operative state. This is shown by some of the grey dots, which follow the black dots.

Post-operative fistulas develop in a high unsaturated fatty acid environment that can damage tissues and make adhesions.

These results demonstrate that one or more lipase inhibitors can be used to treat post-operative pancreatic fistulas.

### Example 3: Treating Post-Operative Pancreatic Fistula

A human identified as having chronic pancreatitis is administered one or more lipase inhibitors (e.g., a composition including one or more lipase inhibitors such as 767) by intraperitoneal injection or intraabdominal injection. The administered inhibitor(s) can reduce the severity of one or more symptoms of chronic pancreatitis.

### Example 4: Preventing Post-Operative Pancreatic Fistula

A human identified as being at risk of developing chronic pancreatitis is administered one or more lipase inhibitors (e.g., a composition including one or more lipase inhibitors such as 767) by intraperitoneal injection or intraabdominal injection. The administered inhibitor(s) can delay or prevent the development of one or more symptoms of chronic pancreatitis.

### Example 5: Treating Post-Operative Pancreatic Fistula

A human identified as having one or more post-operative pancreatic fistulas is administered one or more lipase inhibitors (e.g., a composition including one or more lipase inhibitors such as 767) by intraperitoneal injection or intraabdominal injection. The administered inhibitor(s) can reduce the severity of one or more symptoms of post-operative pancreatic fistula(s).

### Example 6: Preventing Post-Operative Pancreatic Fistula

A human identified as being at risk of developing one or more post-operative pancreatic fistulas is administered one or more lipase inhibitors (e.g., a composition including one or more lipase inhibitors such as 767) by intraperitoneal injection or intraabdominal injection. The administered inhibitor(s) can delay or prevent the development of post-operative pancreatic fistula(s).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for reducing adhesion in a pancreas and/or peritoneal cavity of a mammal, said method comprising administering a lipase inhibitor to said mammal.

2. The method of claim 1, wherein said mammal has chronic pancreatitis, pancreatic cancer, or a post-operative pancreatic fistula.

3. The method of claim 1, wherein said mammal is a human.

4. (canceled)

5. The method of claim 1, wherein said inhibitor comprises the structure: 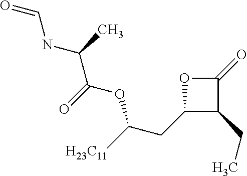
or a pharmaceutically acceptable salt thereof.

6. A method for reducing fibrosis in a pancreas and/or peritoneal cavity of a mammal, said method comprising administering a lipase inhibitor to said mammal.

7. The method of claim 7, wherein said mammal has chronic pancreatitis, pancreatic cancer, or a post-operative pancreatic fistula.

8. The method of claim 6, wherein said mammal is a human.

9. (canceled)

10. The method of claim 6, wherein said inhibitor comprises the structure: 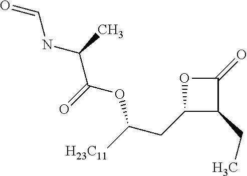
or a pharmaceutically acceptable salt thereof.

11. A method for reducing inflammation in a pancreas of a mammal, said method comprising administering a lipase inhibitor to said mammal.

12. The method of claim 11, wherein said mammal has chronic pancreatitis, pancreatic cancer, or a post-operative pancreatic fistula.

13. The method of claim 11, wherein said mammal is a human.

14. (canceled)

15. The method of claim 11, wherein said inhibitor comprises the structure: 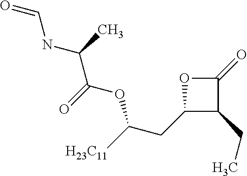
or a pharmaceutically acceptable salt thereof.

16-24. (canceled)

25. A method for improving survival of a mammal, said method comprising administering a lipase inhibitor to said mammal.

26. The method of claim 25, wherein said mammal has chronic pancreatitis, pancreatic cancer, or a post-operative pancreatic fistula.

27. The method of claim 25, wherein said mammal is a human.

28. (canceled)

29. The method of claim 25, wherein said inhibitor comprises the structure: 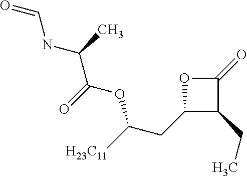
or a pharmaceutically acceptable salt thereof.
